(19) United States
(12) Patent Application Publication
(10) Pub. No.: US 2023/0098121 A1
(43) Pub. Date: Mar. 30, 2023

(54) SYSTEM AND METHOD FOR PROGNOSIS MANAGEMENT BASED ON MEDICAL INFORMATION OF PATIENT

(71) Applicant: Shenzhen Keya Medical Technology Corporation, Shenzhen (CN)

(72) Inventors: Feng Gao, Seattle, WA (US); Hao-Yu Yang, Seattle, WA (US); Yue Pan, Seattle, WA (US); Youbing Yin, Kenmore, WA (US); Qi Song, Seattle, WA (US)

(73) Assignee: Shenzhen Keya Medical Technology Corporation, Shenzhen (CN)

(21) Appl. No.: 17/489,682

(22) Filed: Sep. 29, 2021

(51) Int. Cl. G16H 50/20; G16H 10/60; G16H 50/30; G16H 50/50; G16H 30/40; G06F 16/23; A61B 5/00; A61B 5/02; A61B 5/107

(52) U.S. Cl. CPC G16H 50/20; G16H 10/60; G16H 50/30; G16H 50/50; G16H 30/40; G06F 16/23; A61B 5/4842; A61B 5/7275; A61B 5/02042; A61B 5/1073; A61B 2576/00

(57) ABSTRACT

The disclosure relates to a method for prognosis management based on medical information of a patient, a device, and a medium. The method includes acquiring, by a processor, medical information of the patient at a first time. The method further includes receiving the medical information of the patient at a first time. The method may further include predicting, by a processor, a progression condition of an object associated with the patient at a second time based on the acquired medical information of the first time. The progression condition is indicative of a prognosis risk, and the second time is after the first time. The method may also include outputting the predicted progression condition to an information management system. The method is helpful for users to understand the potential prognosis risk of the object at the second time to aid users in making treatment decisions.

### TECHNICAL FIELD

The present disclosure relates to medical data processing technology, and more particularly, to systems and methods for prognosis management based on medical information of patient.

### BACKGROUND

In the medical field, effective treatments rely on accurate diagnosis and diagnosis accuracy usually depends on the quality of medical image analysis, especially the detection of target objects (such as organs, tissues, target sites, and the like). Compared with conventional two-dimensional imaging, volumetric (3D) imaging, such as volumetric CT, may capture more valuable medical information, thus contributing to more accurate diagnosis. Conventionally, target objects are usually detected manually by experienced medical personnel (such as radiologists), which make it tedious, time-consuming and error-prone.

One such exemplary medical condition that needs to be accurately detected is intracerebral hemorrhage (ICH). ICH is a critical and life-threatening disease, and leads to millions of deaths globally per year. The condition is typically diagnosed using non-contrast computed tomography (NCCT). Intracerebral hemorrhage is typically classified into one of the five subtypes: intracerebral, subdural, epidural, intraventricular and subarachnoid. Hematoma enlargement (HE), namely the spontaneous enlargement of hematoma after onset of ICH, occurs in about one third of ICH patients and is an important risk factor for poor treatment outcomes. Predicting the risk of HE by visual examination of head CT images and patient clinical history information is a challenging task for radiologists. Existing clinical practice cannot predict and assess the risk of ICH patients (for example risk of hematoma enlargement) in an accurate and prompt manner. Accordingly, there is also a lack of accurate and efficient ICH risk management approach.

### SUMMARY

The present disclosure provides a method and device for prognosis management based on medical information of a patient, which may realize automatic prediction for progression condition of an object associated with the prognosis outcome using the existing medical information, so as to aid users (such as doctors and radiologists) in improving assessment accuracy and management efficiency of progression condition of an object, and assist users in making decisions.

In a first aspect, an embodiment according to the present disclosure provides a method for prognosis management based on medical information of a patient. The method may include receiving the medical information of the patient at a first time. The method may further include predicting, by a processor, a progression condition of an object associated with the patient at a second time based on the acquired medical information of the first time. The progression condition is indicative of a prognosis risk, and the second time is after the first time. The method may also include outputting the predicted progression condition to an information management system.

In a second aspect, an embodiment of the present disclosure provides a prognosis management device including an interface and a processor. The interface is configured to receive the medical information of the patient at a first time. The processor is configured to predict a progression condition of an object associated with the patient at a second time based on the acquired medical information of the first time. The progression condition is indicative of a prognosis risk, and the second time is after the first time. The interface is further configured to output the predicted progression condition to an information management system.

In a third aspect, an embodiment of the present disclosure provides a non-transitory computer-readable storage medium having a computer program stored thereon, wherein the computer program, when executed by at least one processor, performs the steps of the method for prognosis management based on medical information of a patient as described above. The method may include receiving the medical information of the patient at a first time. The method may further include predicting, by a processor, a progression condition of an object associated with the patient at a second time based on the acquired medical information of the first time. The progression condition is indicative of a prognosis risk, and the second time is after the first time. The method may also include outputting the predicted progression condition to an information management system.

With the systems and methods for prognosis management based on medical information of a patient according to the disclosed embodiments of the present disclosure, the progression condition of an object at a second time associated with the prognosis outcome may be predicted by using the medical information of the patient at a first time. The predicted prognosis outcome of the second time may be presented to a user to assist the users in learning about the progression condition of the object at the second time. For example, the first time may be a current or past time and the second time is a future time. The predicted future prognosis may aid users such as doctors and radiologists in improving assessment accuracy and management efficiency of progression condition of the object, and assist users in making decisions.

### BRIEF DESCRIPTION OF THE DRAWINGS

In the drawings, which are not necessarily drawn to scale, like reference numerals may describe similar components in different views. Like reference numerals having letter suffixes or different letter suffixes may represent different instances of similar components. The drawings illustrate various embodiments as examples rather than limitations, and together with the description and claims, serve to explain the disclosed embodiments. Such embodiments are demonstrative and not intended to be exhaustive or exclusive embodiments of the present disclosure.

FIG. 1 illustrates an exemplary flowchart of a method for prognosis management according to an embodiment of the present disclosure.

FIG. 2 illustrates an exemplary flowchart for predicting a progression condition of a patient by using a first prediction model according to an embodiment of the present disclosure.

FIG. 3 illustrates another exemplary flowchart for predicting a progression condition of a patient by using a second prediction model according to an embodiment of the present disclosure.

FIG. 4 illustrates an exemplary flowchart for predicting a progression condition of a patient by using a third prediction model that incorporates non-image clinical data of the patient according to an embodiment of the present disclosure.

FIG. 5 illustrates another exemplary flowchart for predicting a progression condition of a patient by using a fourth prediction model that incorporates non-image clinical data of the patient according to an embodiment of the present disclosure.

FIG. 6 illustrates a schematic diagram showing hematoma detection and segmentation by a first portion of a prediction model according to an embodiment of the present disclosure.

FIG. 7 illustrates a schematic diagram showing model training of the first portion of the prediction model of FIG. 6, according to an embodiment of the present disclosure.

FIG. 8 illustrates a schematic diagram showing a prediction flow of a second portion of a prediction model applied to medical information acquired at a single time point, according to an embodiment of the present disclosure.

FIG. 9 illustrates a schematic diagram showing another prediction flow of the second portion of a prediction model applied to medical information acquired at a single time point, according to an embodiment of the present disclosure.

FIG. 10 illustrates a schematic diagram showing yet another prediction flow of the second portion of a prediction model applied to medical information acquired at a single time point, according to an embodiment of the present disclosure.

FIG. 11 illustrates a schematic diagram showing a prediction flow of a second portion of a prediction model applied to medical information acquired at a series of time points, according to an embodiment of the present disclosure.

FIG. 12 illustrates a schematic diagram showing another prediction flow of the second portion of a prediction model applied to medical information acquired at a series of time points, according to an embodiment of the present disclosure.

FIG. 13 illustrates a schematic diagram showing yet another prediction flow of the second portion of a prediction model applied to medical information acquired at a series of time points, according to an embodiment of the present disclosure.

FIG. 14 illustrates a block diagram of a prognosis management device according to an embodiment of the present disclosure.

### DETAILED DESCRIPTION

The disclosure will be described in detail with reference to the drawings and specific embodiments.

As used in this disclosure, words like “first”, “second” do not indicate any particular order, quantity or importance, but are only used to distinguish.

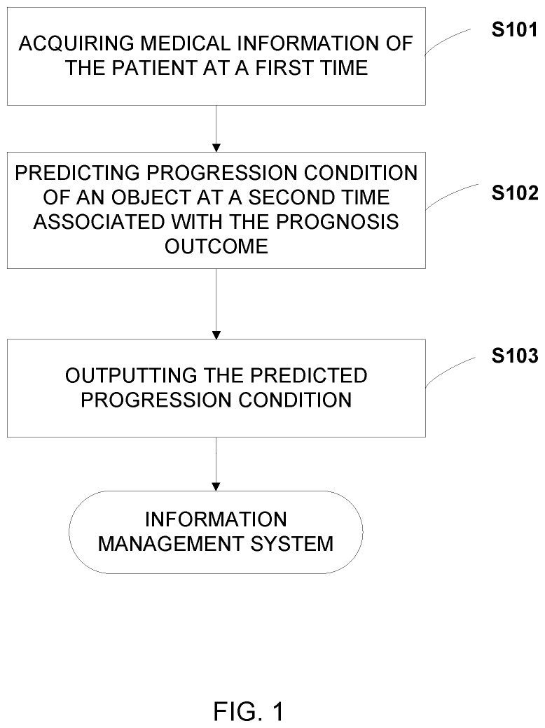

An embodiment of the present disclosure provides a method for prognosis management based on medical information of a patient. As shown in FIG. 1, in step S101, the medical information of the patient at a first time may be acquired by a processor. For example, the medical information of a patient at the first time may be input through a user interface, or may be read from a database, for example, acquired from a local distribution center, or loaded based on a directory of a database. The source from which the medical information of the patient at the first time may be selected does not have specific limitations.

In step S102, the progression condition of an object at a second time associated with the prognosis outcome may be predicted by a processor based on the acquired medical information, where the second time is temporally after the first time. Unlike using the medical information of the patient at current time to perform prediction of the object at the current time, the medical information of the patient at current time is used to predict the progression condition of the object at a certain time in the future, thus facilitating the prognosis management for the patient.

In step S103, the predicted progression condition may be output by the processor for presenting to an information management system. In some embodiments, the information management system may be a centralized system that stores and manages patient medical information. For example, the information management system may store the multi-modality images of a patient, non-image clinical data of the patient, as well as the prognosis prediction results of the patient. The information management system may be accessed by a user to monitor the patient's progression condition. For example, the user may be a doctor. The information management system may include a user interface to provide information to the user or output a prognosis management report to a designated display, or output the predicted progression condition through a text message or multimedia message, or through an applet, etc. The prognosis management method according to the present disclosure can aid the users (e.g., doctors and radiologists) in improving the diagnostic accuracy of the condition of patients, management efficiency of the risks of patients, thus improving the treatment outcome.

Various types of medical information of patients may be utilized, which may be e.g., medical (such as chest X-ray, MRI, ultrasound, etc.) images, medical examining reports, test results, medical advice, etc. The types of medical information of patients are not be specifically limited herein. In some embodiments, the medical information of the patient at the first time includes medical images of the patient at the first time, and optionally non-image clinical data of the patient at the first time. In some embodiments, the medical image may be medical images in DICOM-format, such as CT images, or medical images in other modalities, without limitation. The non-image clinical data may be data such as clinical data, clinical reports, etc. that contain medical information of the patient other than medical images. The medical information may effectively describe the condition of the patient at the first time, so that the progression condition can be predicted quickly through the medical information.

In some embodiments, the medical information of the patient at the first time may include the non-image clinical data of the patient at the first time, which may be acquired from different data sources according to clinical use. For example, in some embodiments, the non-image clinical data can also be acquired from structured clinical information items, or by converting unstructured clinical records into structured clinical information items. As an example, the non-image clinical data may be derived from structured clinical data (such as clinical feature items) or narrative clinical reports, or a combination of both. In some embodiments, if a narrative and unstructured clinical report is provided, it may be converted into structured clinical information items by automated processing methods, such as natural language processing (NLP), according to the required format of the clinical data, to obtain the non-image clinical data. Through this format conversion, various types of data, such as narrative and unstructured clinical reports, etc., may be converted and unified into non-image clinical data which can be processed by a processor, thus reducing the complexity of data processing by the processor.

The prognosis management method of the present disclosure is used to predict the progression condition of the patient at the second time, which is temporally after the first time in the disclosed embodiment. For example, when the second time is a certain time in the future, the first time may be a certain time in the past before the current time or the current time.

In some embodiments, the first time may be a single time point. In an example, one or more medical images of a patient may be captured at a certain time point, then the medical images at that time point can be used as data input of a single time point to perform prediction. Similarly, the clinical report of the patient may be descriptive of the patient at a certain time point, then the clinical report can be used as data input of a single time point to perform prediction. In these examples, medical data of a single time point may be used to predict the progression condition of the object at the second time. That is, the prediction can be performed using a small amount of data, which improves the prediction efficiency and general applicability of the prognosis management method disclosed in this disclosure.

In some alternative embodiments, the first time may be a period of time that includes a series of time points. In an example, several CT images may be captured for a patient at different times within a time period (e.g., a month or a week), then the prediction may be performed according to the CT images of the patient captured at these different time points within the time period. Similarly, medical test reports or clinical reports descriptive of the patient at different time points in the time period may be acquired, so that the medical test reports or clinical reports at different time points may serve as data input to perform prediction. Through using the medical information of a series of time points to predict the prognosis condition of the object at the second time, the accuracy of prediction can be further improved.

In some embodiments, the second time may be a time specified according to the requirement of the user. For example, if the user expects to observe the possible progression condition of the object 3 hours, 4 hours, 12 hours, or even a week or several months after the first time, then the progression condition of the object at the corresponding second time may be predicted and output to the information management system. Alternatively, the second time may be a designated time with a preset interval after the first time, wherein the preset interval may be a time that the doctor deems meaningful to monitor a certain condition, such as 24 hours, 48 hours or 72 hours, so that the user can intuitively get the progression condition of the object at the future time with higher degree of concern, so as to aid the diagnosis of the doctor more efficiently. In some embodiments, the second time may be an arbitrary future time. For example, when predicting hematoma enlargement, the expansion risk of hematoma at an arbitrary future time (any future time dynamically or randomly selected) can be predicted. The enlargement risk of the hematoma in the future is an important reference index for the diagnosis of intracerebral hemorrhage (ICH), which can provide significant guidance for the decision of the doctor.

Various manners may be adopted to set the second time, e.g., setting a fixed time interval between the second time and the first time, or e.g., setting a certain (fixed) time point in the future. In some embodiments, the preset time interval may be adjusted by the processor in response to a user input. In some embodiments, the user may be a doctor, and the doctor may set the second time through inputting instructions. As an example, if the doctor wants to observe the progression condition of a certain disease after 48 hours, but the current output is the progression condition after 24 hours, he/she may input instructions manually to modify the preset time interval between the first time and the second time to 48 hours. Actually, the preset time interval may be dynamically modified as needed by the user. By means of manually adjusting the time interval, the observation requirements of users can be effectively and flexibly satisfied, so that the method for prognosis management disclosed by the invention may be adapted to satisfy a broader range of users in various prognosis management scenarios.

After the preset time interval is determined as well as the medical information of the patient at the first time is received by the processor, the predicted progression condition may be output by the processor, and the specific output may include one or more types of progression conditions. The object may be a patient or an instance of a target object of the patient (hereafter referred to as “an object instance”), such as a hematoma instance. That is, the predicted progression condition output by the processor may be instance-level or patient-level, which may be selected according to the requirement of the user. In some embodiments, the progression condition of an object may include one or more of the following: enlargement risk of an object instance or the patient, deterioration risk of the object instance or the patient, expansion risk of the object instance or the patient, metastasis risk of the object instance or the patient, recurrence risk of the object instance or the patient, location of the object instance, volume of the object instance, and subtype of the object instance.

The object instance may be a site of lesion or a body of lesion in medical image(s). For example, the object instance may be a nodule, a tumor, or any other lesion or medical conditions that may be captured by a medical image. Accordingly, if a patient has nodules, the predicted progression condition of the object in this embodiment can also be the progression condition of the nodules of the patient in the future. Again, the progression condition of the object in this example may include enlargement risk, deterioration risk, expansion risk, metastasis risk, recurrence risk of a nodule or the corresponding patient, location of the nodule, volume of the nodule, and subtype of the nodule, and the like. By means of setting the progression condition associated with the object, the users may learn about the condition of the patient in a more intuitive manner, thus improving the efficiency of diagnosis.

According to the method of the present disclosure, the prognosis management of various diseases may be performed by a processor, for example, the enlargement risk of nodules or tumors may be predicted in the previous example. Based on the principle of the prognosis management scheme disclosed by the present disclosure, the types of diseases targeted are not limited herein. In some embodiments, the object may be a hematoma, and the progression condition includes the enlargement risk of the hematoma for the hematoma instance or the patient, where the first time is after onset of intracerebral hemorrhage (ICH). Hematoma enlargement, namely the spontaneous enlargement of hematoma after onset of ICH, occurs in about one third of ICH patients and is an important risk factor for poor treatment outcomes. By setting the first time after onset of intracerebral hemorrhage, when the hematoma enlargement has occurred, the progression condition of hematoma of the target patient or hematoma instance may be predicted more efficiently based on the medical information (e.g., head CT images and patient clinical history information) at the first time.

The prognosis management method of the present disclosure may predict the progression condition of a patient at a second time by a processor. The processor may be communicatively coupled to a storage and configured to execute computer-executable instructions stored thereon, and the specific process of prediction by the processor is not limited herein. As an example, the progression condition of the object at a second time associated with the prognosis outcome may be predicted by the processor may be performed using a prediction model based on the acquired medical information. As an example, the prediction model may be saved in the storage, and when instructed to perform the prediction, the processor may call the prediction model to perform the prediction of the progression condition of the object at the second time. The prediction model may be pre-trained, and the prediction accuracy of the prediction morel may be further improved as more training data are used for training the prediction model.

The prediction model used by the prognosis management method of the present disclosure may be, for example, a neural network model, a clustering model or a classification model trained as needed, and the specific types of prediction models are not limited herein.

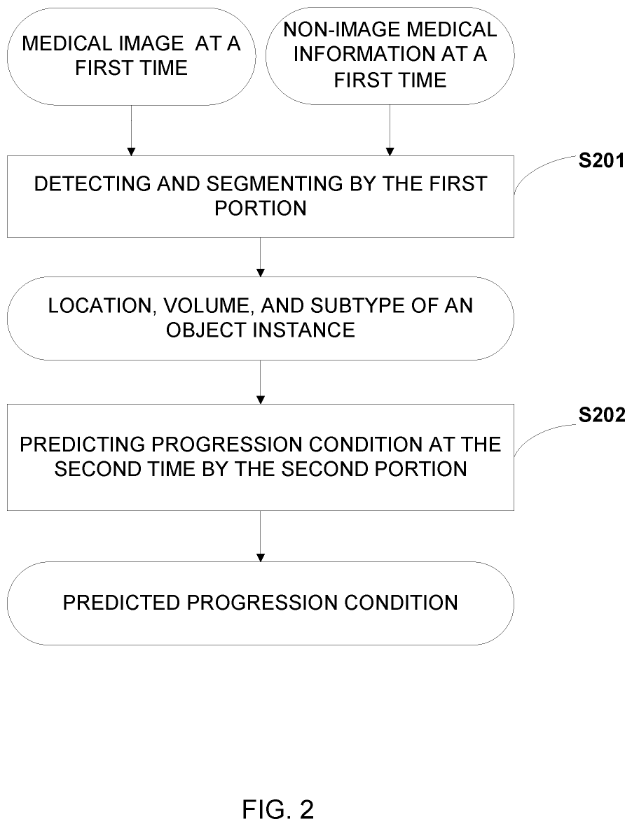

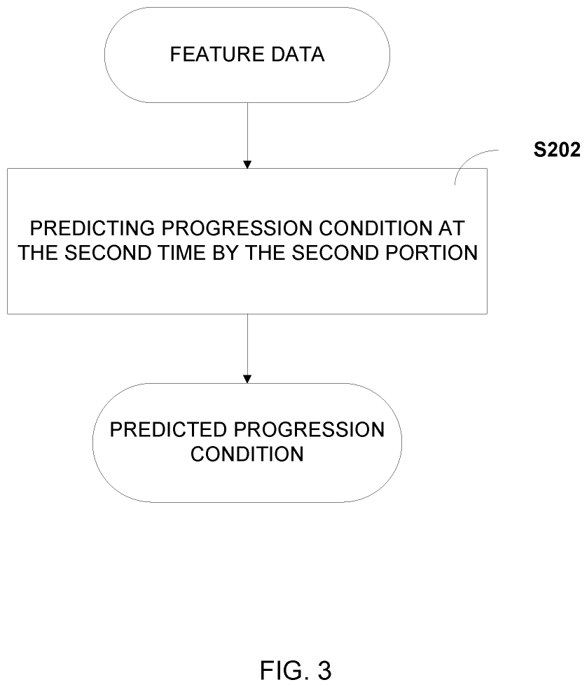

In some embodiments, the prediction model may include a first portion and a second portion, which can be used collectively to predict the progression condition of an object at a second time associated with the prognosis outcome based on the acquired medical information. In step S201, the medical instances may be detected and segmented by the first portion based on medical image of the patient at the first time. After the medical information of the patient at the first time is acquired by the processor, data preprocessing may be performed on the medical information before performing detection and segmentation on the same. As an example, when the object is a hematoma, the non-head images, non-axial images, high-noise images, unwanted clinical information (such as clinical information with too many values missed) may be removed, and filtering operation may be performed on the medical images of the patient, so as to improve the quality of later detection and segmentation. Then, detection and segmentation may be performed by the first portion of the prediction model on the preprocessed medical image of the patient at the first time. By means of detection and segmentation, the position, volume and subtype of each object instance of the patient in the medical image at the first time may be determined, which is then input into the second portion of the prediction model. As shown in FIG. 2, in step S202, the progression condition of each object instance and/or the patient at the second time may be predicted by the second portion based on the output of the first portion. In some embodiments, only the medical image of the patient at the first time is provided by the first portion as a result of the detection and segmentation, to the second portion to perform the prediction. In some examples, as shown in FIG. 3, alternatively or in addition to the output of the first portion (i.e., the results of the detection and segmentation), the feature data deriving from the first portion may be fed into the second portion to perform prediction. Therefore, the second portion of the prediction model can be adjusted according to the type of output provided by the first portion. As a result, the second portion can be trained to predict the progression condition even in absence of medical images of the patient as its input.

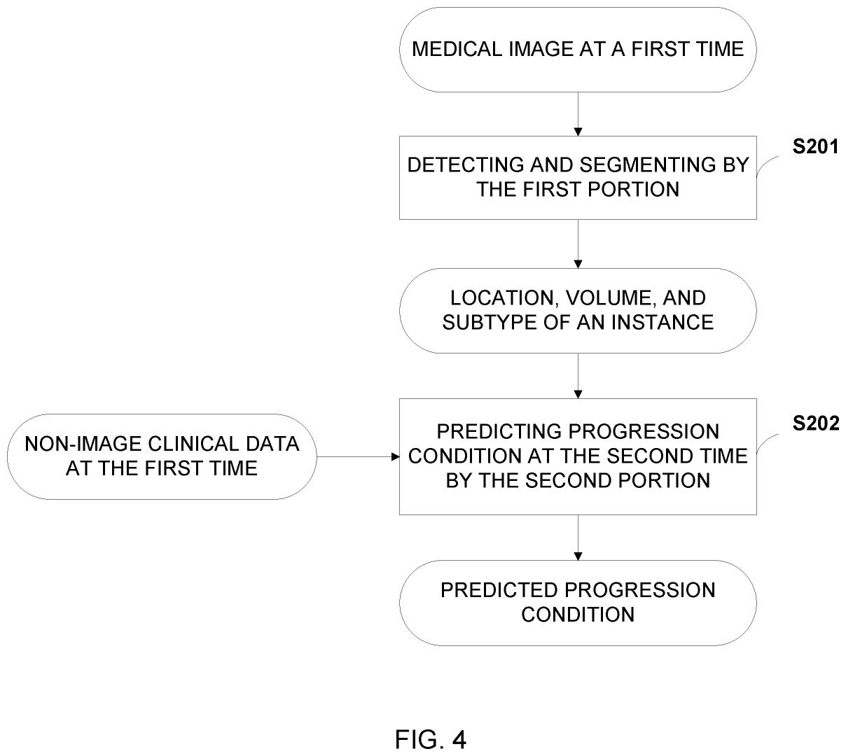

As shown in FIG. 4, the input of the second portion may also alternatively or additionally include non-image clinical data of the patient. As an example, after detecting and segmenting the position, volume and subtype of each object instance of the patient in the medical image at the first time in step S201, the position, volume and subtype of each object instance of the patient in the medical image and the non-image clinical data of the patient at the first time may be input together into the second portion in step S202, so as to predict the progression condition of each instance and/or patient through the second portion. The non-image clinical data of the patient is usually easy to obtain, and the prediction accuracy of the second portion may be improved by combining the non-image clinical data of the patient.

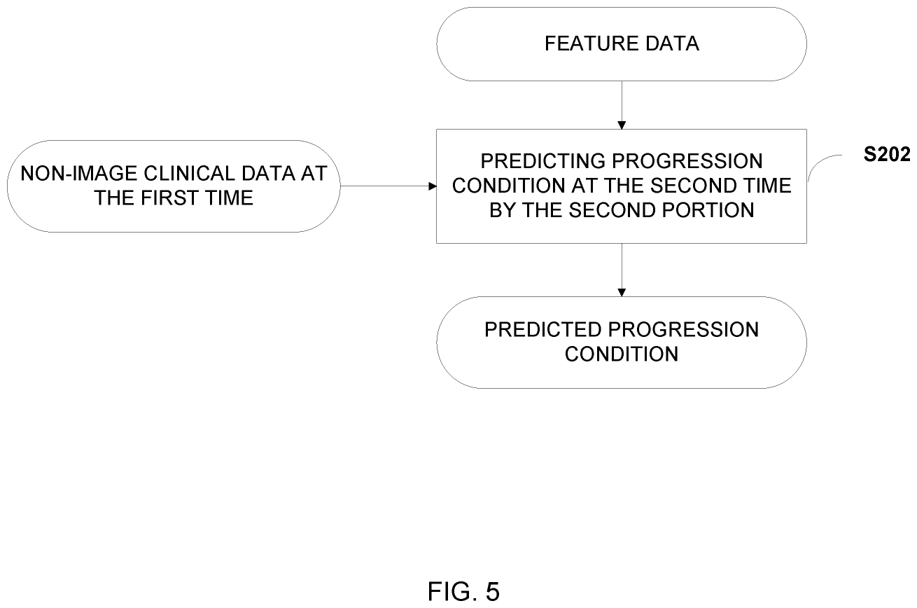

As another embodiment, as shown in FIG. 5, the determined feature data from the first portion may also be input into the second portion together with the non-image clinical data of the patient at the first time for prediction. This embodiment therefore does not require the segmentation or detection result as the input to the first portion. The second portion then predicts the progression condition at the second time at the object instance-level and/or the patient-level. Prediction result may be output at the object instance-level or the patient-level according to the user's preference.

The first portion of the exemplary prediction model may be constructed based on a specified target detection framework, which may be based on a neural network of any suitable structure. As an example, the first portion may be constructed based on a mask RCNN, and configured to determine the location, volume and subtype for each object instance. In this example, by determining the location, volume and subtype of each object instance, the object may be located accurately, thus improving the speed of prediction.

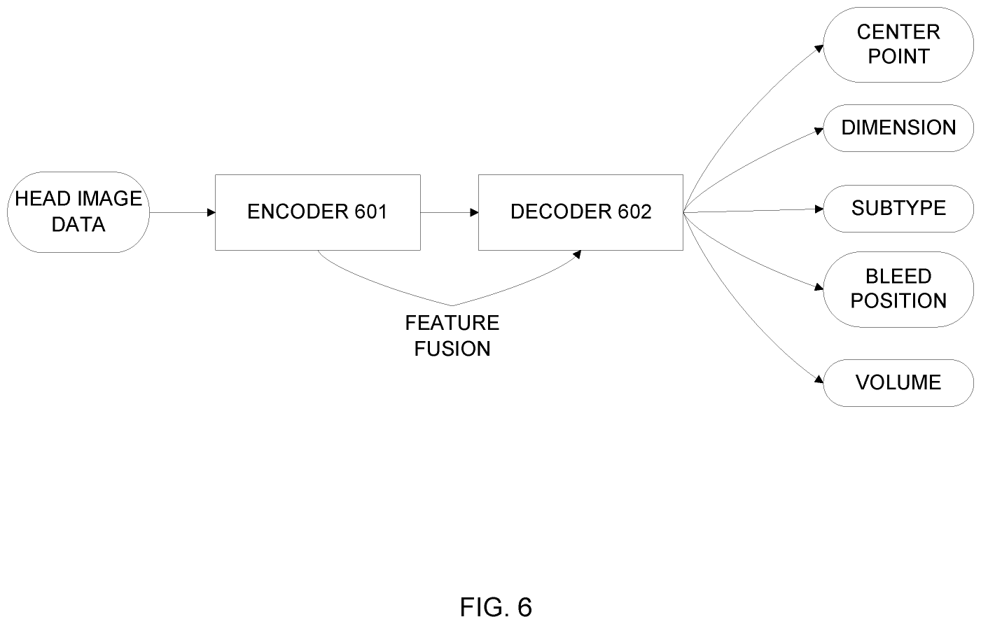

In case that the object is a hematoma, the first portion may also be configured to determine the center point, size, subtype, bleed position and volume of each hematoma instance. As shown in FIG. 6, the mask RCNN may be a multi-task encoder-decoder network, which may be used to perform voxel-level classification tasks and regression tasks. In this example, detection and segmentation may be performed by the trained mask RCNN on the medical images related to the hematoma, to obtain the center point, size, subtype, bleed position and volume of each hematoma instance. As an example, in FIG. 6, the head image data of the hematoma patient may be input into the encoder 601 of the mask RCNN, and then the output of the encoder 601 may be used as the input of the decoder 602, to detect the hematoma instance in the image and segment the instance. Based on the bleed position, center point and size of each hematoma instance, a bounding box of the hematoma instance may be determined. Then, in step S202, the center point, size, subtype, bleed position and volume may be used as the input of the second portion to perform the prediction. The results of the specific detection and segmentation from the first portion, may be adjusted by the doctors with a focus depending on the disease types.

An exemplary training process of the mask RCNN adapting for the hematoma will be described. It is contemplated other implementation methods may also be adopted for the training process.

In some embodiments, the following training labels may be used: (i) a center point heatmap, e.g., a probability heatmap for each voxel being a center point in medical image; (ii) a voxel-level subtype label; (iii) a hematoma dimension (height, width and depth) assigned to each voxel according to the hematoma it belongs to, (iv) a hematoma volume (by counting the hemorrhage voxels and rescale using voxel dimension), and (v) a bleed position assigned to each voxel according to the hematoma it belongs to. For training processes performed to train the mask RCNN for different types of diseases other than hemorrhage enlargement, the types of training labels may be adapted according to the different concerns of doctors.

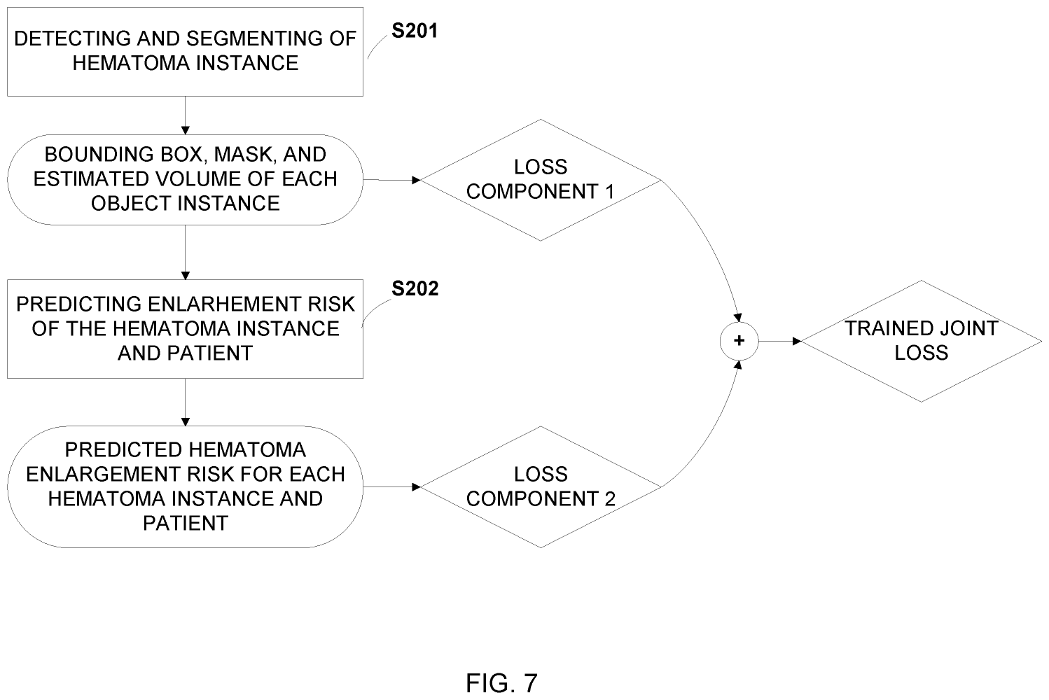

As shown in FIG. 7, the training labels may be obtained as the detection and segmentation results using the mask RCNN architecture. In some embodiments, the mask RCNN architecture may be trained using a joint loss taking into account these training labels collectively. In this example, the joint loss function for training the mask RCNN may be the weighted sum of aforementioned labels, e.g., according to the following Equation (1):

[symbol]

=ω_{ctr} [symbol] center+ω_{dim} [symbol] _{dim}+ω_{sub} [symbol] _{sub}+ωₚₒₛ [symbol] ₚₒₛ+ωᵥₒₗ [symbol] ᵥₒₗ (1)

where [symbol] represents the joint loss, [symbol] _{center} is the loss corresponding to the center point label, [symbol] _{dim} is the loss corresponding to the hematoma dimension label, [symbol] ᵥₒₗ is the loss corresponding to the hematoma volume label, [symbol] _{sub} is the loss corresponding to the voxel-level subtype label, and [symbol] ₚₒₛ is the loss corresponding to the bleed position label. ω_{ctr} is the weight corresponding to the center point label, ω_{dim} is the weight corresponding to the hematoma dimension label, ω_{sub} is the weight corresponding to the voxel-level subtype label, ωₚₒₛ is the weight corresponding to the bleed position label, and ωᵥₒₗ is the weight corresponding to the hematoma volume label. [symbol] _{sub} and [symbol] ₚₒₛ take the form of cross entropy loss, while [symbol] _{center}, [symbol] _{dim} and [symbol] ᵥₒₗ may take the form of ι₂ loss.

During the prediction stage, center points may be determined by an optimal network threshold tuned in the training stage. After center points are obtained, the dimension output from associated center point voxels is utilized to determine the region of hematoma instance (namely bounding boxes of hematoma instances may be generated), while the subtype masks are utilized to determine the hemorrhage voxels. The hematoma volume may be calculated by counting the hemorrhage voxels and rescaling the same using voxel dimension. The bleed position may also be obtained from voxel-level labels. The hematoma bounding boxes are then refined using non-maximum suppression.

In some embodiments, the second portion of the prediction model may be constructed as a machine learning model or a deep learning model.

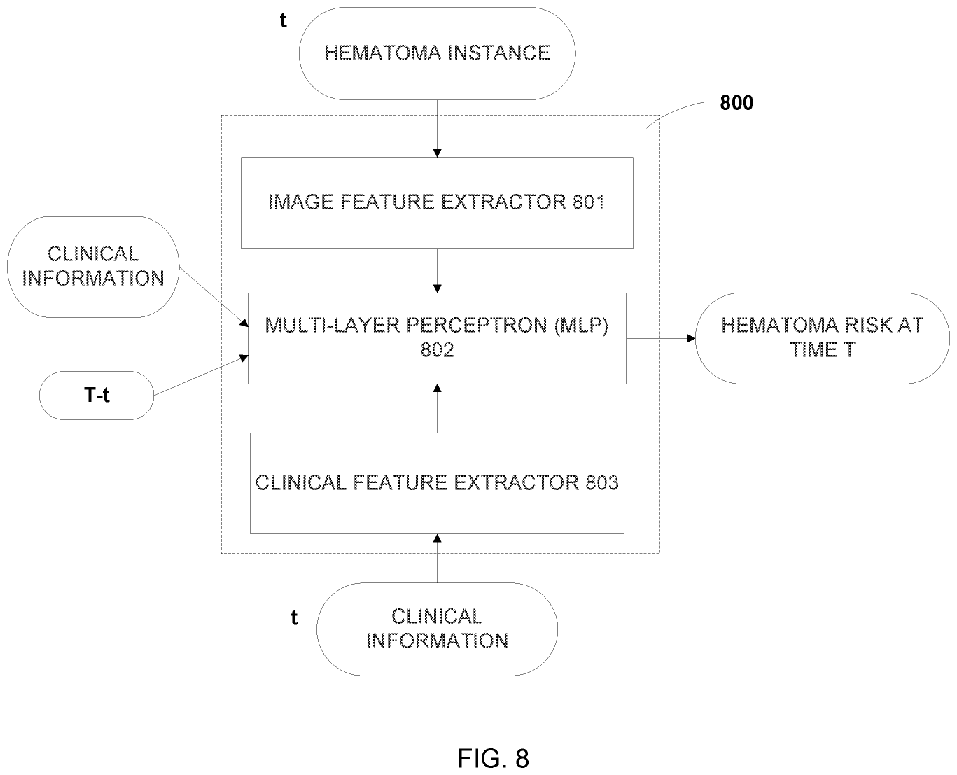

For example, as shown in FIG. 8, the second portion 800 may be configured to extract image features of object instances. The second portion 800 may include a multi-layer perceptron (MLP) 802, which may predict the progression condition of each object instance and/or patient at the second time based on the extracted image features and the delay period between the first time and the second time. FIG. 8 illustrates a schematic diagram showing a prediction flow when the second portion 800 includes the multi-layer perceptron 802. Take the hematoma as an example of object, as shown in FIG. 8, for example, the prediction of the risk of hematoma enlargement at time T of the patient is desired. The input of the multi-layer perceptron 802 may include the image features of the hematoma instances extracted by the image feature extractor 801. In this example, the image feature extractor 801 for single time point may be implemented by using a convolutional neural network (CNN). As an example, in FIG. 8, a preset time interval (T-t) between the first time t and the second time T may also be input into second portion 800. Clinical feature data extracted from non-image clinical data by the clinical feature extractor 803 (e.g., implemented using MLP) may also be input into second portion 800. In practical applications, a single MLP network may be utilized to extract clinical feature data from non-image clinical data and predict the progression condition of each object instance and/or patient at the second time, or separate networks may be utilized for the extraction process of clinical feature data and the prediction process of progression condition, which may be specifically determined according to actual conditions. Other image features of the hematoma instances may also be used as the input of the multi-layer perceptron 802 altogether. As an example, other image features may be the blood position and volume of the hematoma instance at time t. Therefore, the risk of hematoma enlargement at time T can be predicted by the trained multi-layer perceptron. With regard to the training of the second portion 800, the parameters of the multi-layer perceptron may be optimized by a common optimizer. For example, when the first time is a single time, the loss function of the multi-layer perceptron may be a binary entropy loss. Other applicable models of the second portion 800 may also be trained by similar algorithms. In this example, through the design of the second portion 800, accurate predictions can be made even when the data input is only the medical information of the patient at a single time point, thereby effectively assisting doctors to further improve the diagnostic accuracy of the condition of the patients.

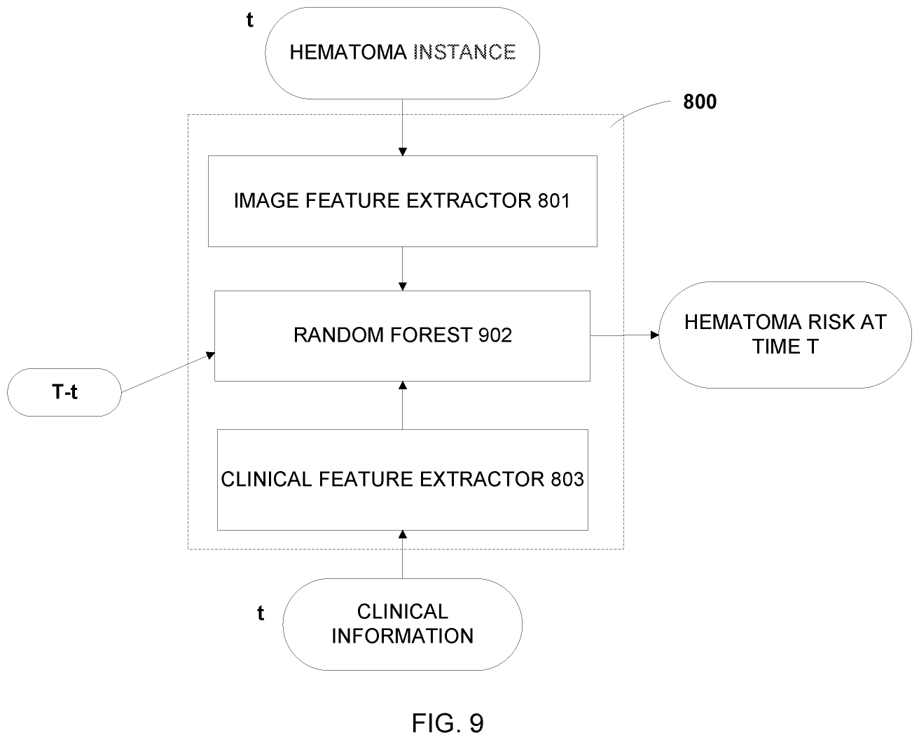

As another example, the second portion 800 may alternatively include a random forest 902 instead of multi-layer perceptron 802. As shown in FIG. 9, the input of the random forest 902 may also include aforementioned various data, which will not be repeated in detail herein. The prediction of the risk of hematoma enlargement at time T may also be performed without introducing data such as other image features.

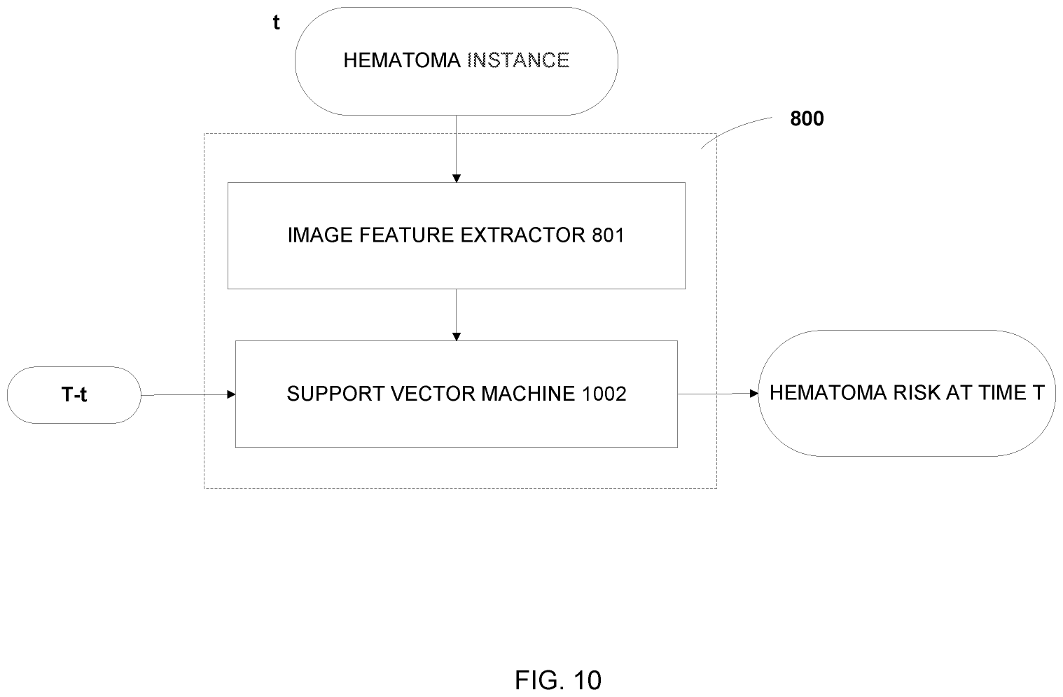

The second portion 800 may alternatively include a support vector machine (SVM) 1002. As shown in FIG. 10, the input of the SVM 1002 may also contain the aforementioned various data, which will not be repeated in detail herein. As an example, in FIG. 10, the prediction may be implemented only by inputting the image features of the hematoma instances extracted by the image feature extractor 801 and the preset time interval between the first time t and the second time T.

The above is only an example of hematoma enlargement risk prediction, and the types of data input to random forest, SVM or MLP are not limited. In practical applications, the feature data input to the second portion 800 may be adjusted as needed.

The prognosis management method according to the present disclosure described above is applicable to the medical information prediction process in which the medical information of a patient at the first time acquired by a processor is medical information at a single time point. As an example, the prediction may be performed based on the medical image of a single time point, or based on the medical image of a single time point in combination with the non-image clinical data of the single time point.

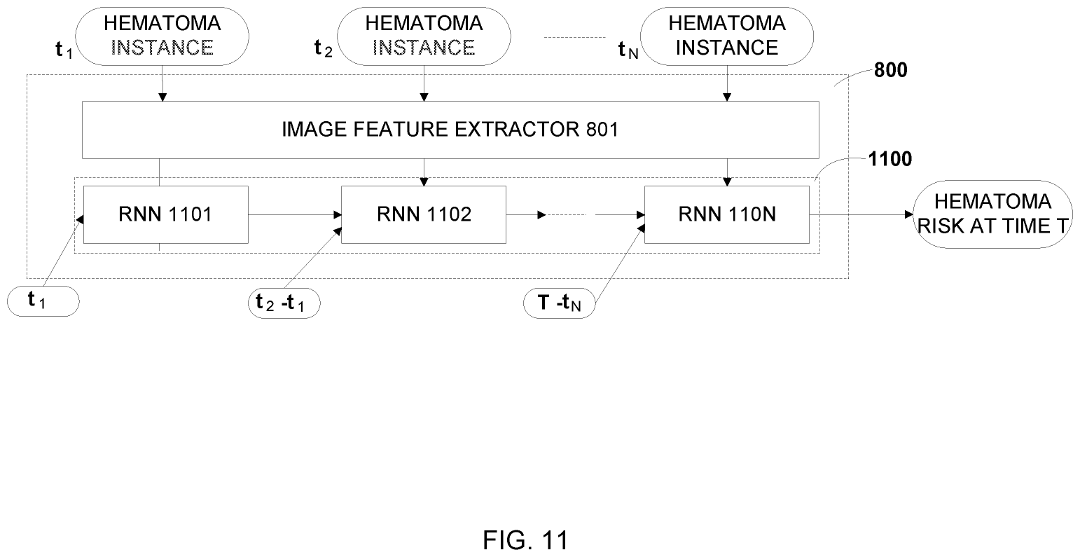

In some embodiments, the prediction may be performed by the second portion in case that the first time includes a series of time points. Take the hematoma as an example of the object again, and the prediction with respect to other types of objects may refer to this prediction process. When the first time includes a series of time points, for example, the first time includes N time points, t₁, t_{N}, as shown in FIG. 11, the corresponding second portion 800 may include a series of RNN units 1100 corresponding to the time points, for example, RNN unit 1101 to RNN unit 110n. The RNN units in this example may be selected from, for example, a long-short-term memory neural (LSTM) network or a gated recurrent unit (GRU), suitable for processing sequential data. For example, in FIG. 11, the second portion 800 may be constructed as a prediction model including N RNN units. Therefore, the image features of each object instance at time points of t₁, t_{N} may be extracted by the image feature extractor 801 of the second portion 800. Particularly, the image feature extractor 801 of the second portion 800 may be constructed based on CNN with the same structure as that of the image feature extractor used for the single time point mentioned above, and may also be constructed by CNN with other structures. The hematoma risk of each object instance and/or patient at time T may be predicted by a series of RNN units. As shown in FIG. 11, the input of each RNN unit 110i (i=1, 2, . . . ,N) may include: the extracted image features of the object instance at the corresponding time point the output of the adjacent upstream RNN unit, and the time interval (tᵢ-tᵢ₋₁) between the corresponding time point t₁ and the corresponding time point tᵢ₋₁ of the adjacent upstream RNN unit. For example, in FIG. 11, the time interval input to the first RNN unit 1101 may be set as the time point t₁. Through a series of RNN units, the feature data of a series of time points may be introduced into the next RNN unit, so that the hematoma risk of each object instance and/or patient at time T may be output through the last RNN unit 110n. When the first time includes a series of time points, all available training labels from different time points may be used for training. The loss function used for training the RNN units may be a joint loss of these different time points. For example, the loss function of the second portion 800 may take the form of Equation (2):

[symbol]

=Σₜωₜ [symbol] ₜ (2)

where [symbol] ₜ is the loss function for time t, which may be a binary entropy, and ωₜ is a weight factor.

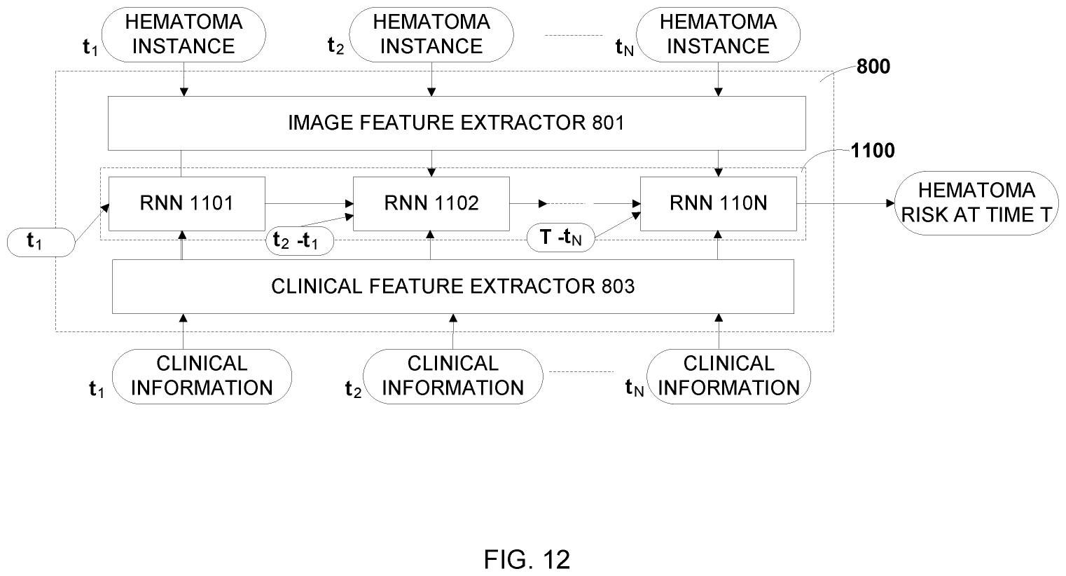

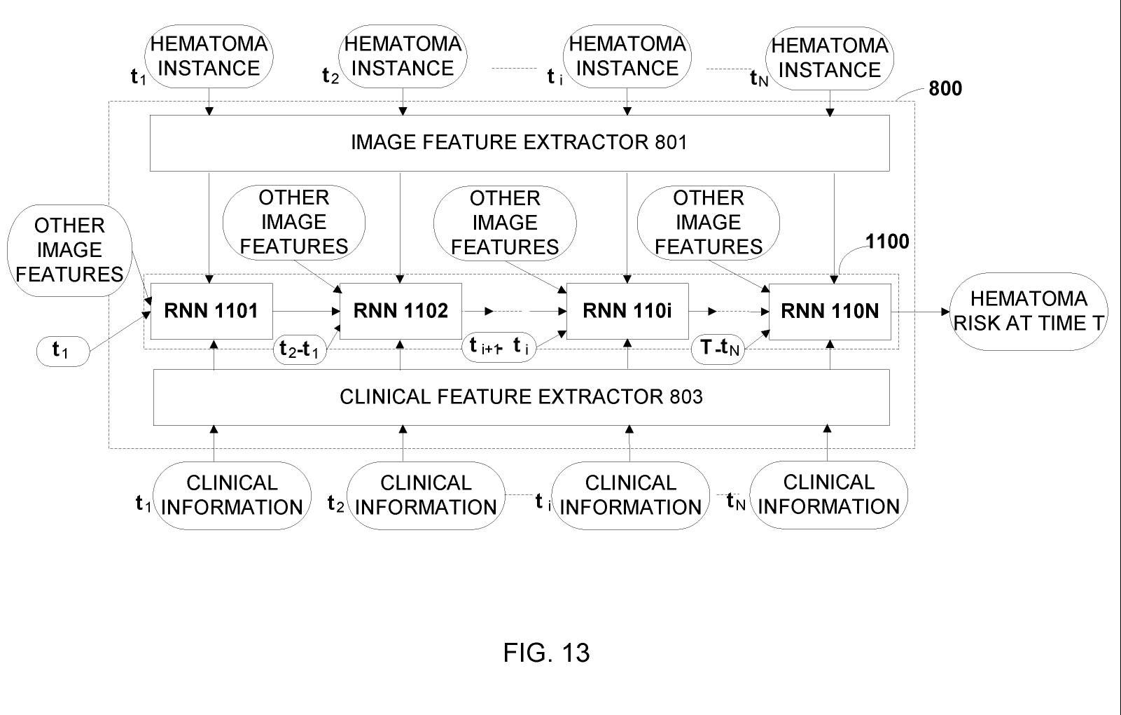

In case that the medical information of the patient at the first time further includes a series of non-image clinical data, the clinical features corresponding to the non-image clinical data at time points of t₁, . . . , t_{N} may be extracted by the clinical feature extractor 803 of the second portion 800. The clinical feature extractor 803 may also be constructed based on an MLP with the same structure as the clinical feature extractor used for the single time point mentioned above. The clinical characteristics of each time point of t₁, t_{N} may be taken as another group of inputs to the corresponding RNN units, as shown in FIG. 12, so that the prediction may be performed by using the medical images of patients at a series of time points and the corresponding non-image clinical data at the series of time points. As shown in FIG. 13, the input of each RNN unit 110i may also include other image features at this time point e.g., the blood position and volume of the hematoma instance at etc. The other image features may be used as an optional input of RNN unit, which is not limited herein. When medical information of the patient at a series of time points can be obtained, by using a plurality of series of RNN units in the second portion, the medical information of the patient at the plurality of time points may propagate downstream in the direction of sequence. As a result, the input of the subsequent RNN units may contain the condition characteristics of the patient at the previous time points, which may effectively improve the prediction accuracy of the second portion and provide doctors with more accurate prediction results.

Finally, the predicted progression condition may be output by the processor to the information management system and may be presented to the user. As an example, different approaches can be applied, such as taking the maximum risk score across all the instances, to achieve the effect of combining instance-level risk scores into patient-level risk scores. For example, the optimal probability score cut-off may be selected by using the ROC curve (receiver operating characteristic curve). Besides, the probability score can also be converted into a binary decision (e.g., the hematoma will enlarge or not, or the patient will suffer from HE or not). The prediction process can be adapted for other progression conditions.

The prognosis management method disclosed by the present disclosure adopts a prediction model to predict the progression condition based on the acquired medical information of the patient. Firstly, it may predict the progression condition of the patient or the object stance at a certain time point in future based on medical information of the patient as little as at a single time point. Therefore, the disclosed method can provide efficient assistance for the diagnosis by the doctors with a low amount of data. Further, by means of specific design of the prediction model, it may be further adapted to prediction based on medical information of the patient at a series of time points. Taking the series of medical information as input, the prediction accuracy of the model may be further improved and thus improving the aiding effect for diagnosis by doctors.

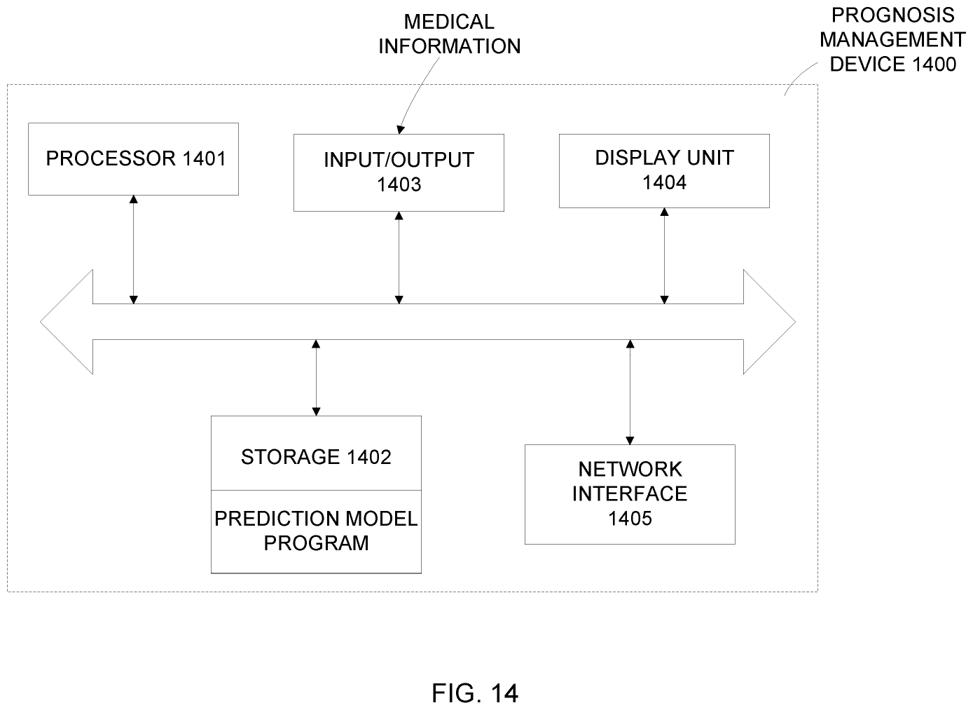

The embodiment of the present disclosure also provides a prognosis management device, as shown in FIG. 14. The prognosis management device 1400 may include a processor 1401, a storage 1402 and a communication bus. The communication bus may be used to realize connection and communication between the processor 1401 and the storage 1402. The processor 1401 may be a processing device including one or more general-purpose processing devices such as a microprocessor, a central processing unit (CPU), a graphics processing unit (GPU), and the like. More specifically, the processor may be a complex instruction set computing (CISC) microprocessor, a reduced instruction set computing (RISC) microprocessor, a very long instruction word (VLIW) microprocessor, a processor running other instruction sets, or a processor running a combination of instruction sets. The processor can also be one or more dedicated processors specialized for specific processing, such as an application specific integrated circuit (ASIC), a field programmable gate array (FPGA), a digital signal processor (DSP), a system on a chip (SoC), and the like. In some embodiments, the prognosis management device 1400 may further include an input/output 1403, which is also connected to the communication bus. The input/output 1403 may be used for the processor 1401 to acquire externally input medical information of the patient, and the input/output 1403 may also be used to input the medical information of the patient into the storage 1402. As shown in FIG. 14, a display unit 1404 may also be connected to the communication bus, and the display unit 1404 may be used to display the operating process of the prognosis management device and/or the output of the prediction result. The processor 1401 may also be used to execute one or more computer programs stored in the storage 1402, for example, a prediction model program may be stored in the memory, and executed by the processor 1401 to perform the steps of the method for prognosis management based on medical information of patients according to various embodiments of the present disclosure.

Embodiments of the present disclosure also may provide a non-transitory computer-readable storage medium having a computer program stored thereon, wherein the computer program, when executed by at least one processor, performs the steps of the method for prognosis management based on medical information of a patient according to any embodiment of present disclosure. A computer-readable medium may be a non-transitory computer-readable medium such as a read only memory (ROM), a random access memory (RAM), a phase change random access memory (PRAM), a static random access memory (SRAM), a dynamic random access memory (DRAM), an electrically erasable programmable read only memory (EEPROM), other types of random access memory (RAM), a flash disk or other forms of flash memory, a cache, a register, a static memory, a compact disc read-only memory (CD-ROM), a digital versatile disc (DVD) or other optical memory, a cassette tape or other magnetic storage device, or any other possible non-transitory medium used to store information or instructions that can be accessed by computer devices, and the like.

In addition, although exemplary embodiments have been described herein, the scope thereof includes any and all embodiments having equivalent elements, modifications, omissions, combinations (for example, schemes in which various embodiments intersect), adaptations or changes based on the present disclosure. The elements in the claims will be broadly interpreted based on the language adopted in the claims, and are not limited to the examples described in this specification or during the implementation of this application, and the examples thereof will be interpreted as non-exclusive. Therefore, the embodiments described in this specification are intended to be regarded as examples only, with the true scope and spirit being indicated by the following claims and the full range of equivalents thereof

What is claimed is:

1. A method for prognosis management based on medical information of a patient, comprising:
receiving the medical information of the patient at a first time;
predicting, by a processor, a progression condition of an object associated with the patient at a second time based on the received medical information of the first time, wherein the progression condition is indicative of a prognosis risk, wherein the second time is after the first time; and
outputting the predicted progression condition to an information management system.

2. The method of claim 1, wherein the medical information of the patient at a first time includes medical images of the patient at the first time or non-image clinical data of the patient at the first time.

3. The method of claim 1, wherein the first time includes a single time point or a series of time points.

4. The method of claim 1, wherein, the second time is an arbitrary future time or a specified time with a preset time interval after the first time.

5. The method of claim 4, further comprising: adjusting the preset time interval, by the processor, in response to a user input.

6. The method of claim 1, wherein the prognosis risk includes at least one of a enlargement risk of the object, a deterioration risk of the object, an expansion risk of the object, a metastasis risk of the object, a recurrence risk of the object, a location of the object, a volume of the object, and a subtype of the object.

7. The method of claim 1, wherein the object includes a hematoma, and the prognosis risk includes an enlargement risk of the hematoma for the hematoma, and the first time is after onset of an intracerebral hemorrhage.

8. The method of claim 2, wherein the non-image clinical data is obtained from structured clinical information items or obtained by converting unstructured clinical records into structured clinical information.

9. The method of claim 2, wherein predicting the progression condition of the object at the second time based on the received medical information comprises applying a prediction model to the received medical information, wherein the prediction model is a deep learning model trained to predict the progression condition.

10. The method of claim 9, wherein the prediction model includes a first portion and a second portion, and predicting the progression condition of the object at the second time further comprises:
detecting and segmenting the object by the first portion from the medical image of the patient at the first time, wherein the first portion further extracts features from the medical information of the patient; and
predicting the progression condition of the object at the second time by the second portion based on the segmented object or the features determined by the first portion, or the non-image clinical data of the patient.

11. The method of claim 10, wherein the first portion includes a multi-task encoder-decoder network and configured to determine a location, volume, and subtype of the object.

12. The method of claim 10, wherein the object is a hematoma, and the first portion is configured to determine a center point, dimension, subtype, bleed position, and volume of the hematoma.

13. The method of claim 10, wherein the first time is a single time point, and the second portion includes an MLP and configured to extract image features of the object and predict the progression condition of the object at the second time based on the extracted image features and a time interval between the first time and the second time.

14. The method of claim 13, wherein the second portion is further configured to extract non-image features from the non-image clinical data of the patient, and predict the progression condition of the object at the second time based on the extracted image features, the extracted non-image features and the time interval.

15. The method of claim 10, wherein the first time includes a series of time points, the second portion includes a series of RNN units corresponding to the series of time points, and the second portion is configured to extract image features of the object at the series of time points, and predict the progression condition of the object at the second time, wherein each RNN unit is applied on the extracted image features of the object at the corresponding time point, the output of an adjacent upstream RNN unit, and a time interval between its corresponding time point and the time point corresponding to the adjacent upstream RNN unit, and the last RNN unit is configured to output the progression condition of the object at the second time.

16. The method of claim 15, wherein the second portion is further configured to extract non-image features from the non-image clinical data of the patient at the series of time points, and each RNN unit is further applied on the extracted non-image features at the corresponding time point.

17. A prognosis management device, comprising:
an interface configured to receive medical information of a patient at a first time; and
a processor configured to predict a progression condition of an object associated with the patient at a second time based on the received medical information of the first time, wherein the progression condition is indicative of a prognosis risk, wherein the second time is after the first time,
wherein the interface is further configured to output the predicted progression condition to an information management system.

18. The prognosis management device of claim 17, wherein the object includes a hematoma, and the prognosis risk includes an enlargement risk of the hematoma for the hematoma, and the first time is after onset of an intracerebral hemorrhage.

19. The prognosis management device of claim 17, wherein to predict the progression condition of the object at the second time based on the received medical information, the processor is configured to:
detect and segment the object by a first portion of a prediction model from a medical image of the patient at the first time, wherein the first portion further extracts features from the medical information of the patient; and
predict the progression condition of the object at the second time by a second portion of the prediction model based on the segmented object or the features determined by the first portion.

20. A non-transitory computer-readable storage medium having a computer program stored thereon, wherein the computer program, when executed by at least one processor, performs a method for prognosis management based on medical information of a patient, the method comprising:
receiving the medical information of the patient at a first time;
predicting a progression condition of an object associated with the patient at a second time based on the received medical information of the first time, wherein the progression condition is indicative of a prognosis risk, wherein the second time is after the first time; and
outputting the predicted progression condition to an information management system.
